(19) United States
(12) Patent Application Publication
(10) Pub. No.: US 2003/0219884 A1
(43) Pub. Date: Nov. 27, 2003

(54) Method for carrying out electrical measurements on biological membrane bodies

(75) Inventors: Frank Lison, Ganting (DE); Christoph Methfessel, Wuppertal (DE); Katrin Schnizler, Nurtingen (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(73) Assignee: Bayer Aktiengesellschaft, Leverkusen (DE)

(21) Appl. No.: 10/354,530

(22) Filed: Jan. 30, 2003

(30) Foreign Application Priority Data
Jan. 31, 2002 (DE) 10203686.1

(51) Int. Cl. C12N 13/00

(52) U.S. Cl. 435/173.1

(57) ABSTRACT

The present invention relates to a method for carrying out electrical measurements on biological membrane bodies, which are located on a substrate. The electrically conductive access to the membrane body, which is needed for the measurement, is made in situ by intense laser beams.

### METHOD FOR CARRYING OUT ELECTRICAL MEASUREMENTS OF BIOLOGICAL MEMBRANE BODIES

The present invention relates to a method for carrying out electrical measurements on biological membrane bodies, which are located on a substrate. The electrically conductive access to the membrane body, which is needed for the measurement, is made in situ by intense light beams.

Ion channels and receptors are gaining importance as active-agent targets in the pharmaceutical industry, crop protection and animal health. In particular by decoding the human genome and the genomes of other organisms, active-agent research is provided with a large number of new, potential active-agent targets. In many cases, these are novel ion channels or new subunits of already know ion channels. In order to identify active agents which modulate ion channels, so-called electrophysiological measurements are indispensable. In this case, the current of electrically charged particles flowing through the ion channels is directly measured.

The state of the art for studying ion channels in cell membranes is currently the patch-clamp method, which was developed in the mid-1970s by Neher and Sakmann (O. P. Hamill et al., Pflügers Arch. 391, 85 (1981)). Using this method, the current through individual ion channels could be measured for the first time, and even quite small cells could now be characterized electrophysiologically. In contrast to the previously known methods for large cells, in which a glass capillary or pipette filled with an electrolyte is inserted into the cell, in the patch-clamp method the glass capillary is placed carefully on the cell membrane and slight suction is applied to the latter. This forms the so-called gigaseal, an extremely high-impedance, electrically leaktight connection between the pipette tip and the cell membrane. It isolates a small membrane spot, the “patch”, from the rest of the cell surface and hence makes it possible to observe individual ion channels in this patch. By using a negative-pressure pulse or an electrical pulse or suitable chemical substances, the cell-membrane patch isolated below the pipette can be broken down, and a high-quality electrical access to the cell interior can hence be established. A high-quality electrical access is a low-impedance (<20 megaohm) connection between the interior of the pipette and the interior of the cell, with a negligible (in the upper megaohm range, preferably in the gigaohm range) parallel resistance between the interior of the pipette and the electrolyte solution surrounding the cell. In other regards, the cell is not damaged or broken down during this. In this so-called whole-cell electrical lead-off, the activity of ion channels in the entire membrane of the cell is observed simultaneously. Thanks to the high quality of the electrical lead-off, currents of up to a few picoamperes can be drawn off precisely with measurement times of a few milliseconds, so as to obtain an almost perfect, detailed picture of the activity of ion channels under the influence of arbitrary, freely selectable measurement conditions, for example the voltage, temperature, active-agent delivery, intracellular or extracellular composition of the salt solution, etc.

A crucial disadvantage of the patch-clamp method for use in industrially finding active agents is the elaborate preparation for the measurements, which allows even experienced electrophysiologists only about 20 measurements per day. Furthermore, the conventional patch-clamp technique requires great experience and much manual dexterity. Various study groups and companies are therefore working on automating or parallelizing the patch-clamp method or another comparable measurement arrangement.

WO 00/34776, WO 96/13712 and WO 98/50791 disclose concepts for automating the prior patch-clamp method with glass pipettes. In this case, some or all of the elaborate manual working steps are carried out by machine under the control of a computer. This approach reduces the experimenter's workload and increases the number of measurements carried out by a certain factor, although generally not more than ten times. This type of automation is furthermore technically very demanding and requires extensive equipment, and it is not parallelizable to a sufficient degree.

WO 98/22819, WO 01/27614, WO 99/66329, WO 01/25769, DE 199 36 302, EP 0 962 524, EP 0 960 933, WO 99/28037, WO 99/31503, WO 01/48474, WO 01/48475, WO 01/59447 disclose further methods and devices for automating the patch-clamp technique, in which the patch-clamp pipette is replaced by a planar or microstructured substrate with a suitable opening. For example, this may be a membrane or thin film, which has been provided with holes measuring only a few micrometres in size. The described methods and devices are then based on the cells occupying the holes and on a seal being formed there, which is similar to the gigaseal in the case of the patch pipette. This occupation of the holes by the cells may take place by random movement of the cells, or it may be induced or supported by a pressure gradient, by inhomogeneous electric fields, using so-called optical tweezers, or by other forces on the cells. After a cell has successfully occupied an aperture, so that the aperture is closed in an electrically leaktight manner by the membrane of the cells, an electrically conductive access through the aperture into the interior of the cell is obtained by opening the cell membrane, which may be done chemically, by pressure or electrically. In the described methods and devices, the required electrically conductive connection to the interior of the cell is hence obtained, and electrophysiological measurements can subsequently be carried out. Owing to the planar arrangement and the fundamental possibility of filling a plurality of holes in a substrate with cells in parallel, the throughput of these devices and methods can be increased. A crucial feature of all these methods and devices is that the substrates have one or more suitable apertures and, for measurement, the cells need to be placed precisely on the aperture.

Both described concepts for automating the patch-clamp method function only with cells which are in suspension. In the case of adherently growing cell lines, the cells firstly need to be detached from the substrate. This may be done either mechanically, by using a suitable salt solutions (for example calcium-free) or by means enzymes, and it generally leads to great stress on the cells.

The state of the art for materials processing with laser beams involves laser ablation methods, in which the desired structures are made in the materials to be structured by means of intense, generally highly focussed laser beams, preferably pulsed lasers or short-pulse lasers. By focussing the laser beams, intensities in the range of gigawatts or even terawatts per square centimetre can be achieved at the focus, so that chemical bonds are broken. Lasers which are employed are both continuous-wave and pulsed light sources in the UV range, for example excimer lasers, nitrogen lasers as well as short-pulse and ultrashort-pulse lasers in the visible and near-infrared ranges, in particular titanium-sapphire lasers. The typical structure sizes which can be achieved with these methods depend on the wavelength, the laser which is used, the material properties and the focus diameter. It is known from P. Simon et al. (Photonics Sci News 5, 59 (1999)) and S. Nolte et al. (Nonlinear Opt. 24, 229 (2001)) that laser ablation can be used to produce structures with a lateral dimension of less than one micrometre which have a high aspect ratio, the aspect ratio being given by the ratio between the height and the width of the structure.

It is furthermore known prior art that these laser ablation methods can also be used to process biological materials and living tissue, in particular including cells and their cell membranes. Besides VUV and UV lasers, short-pulse lasers in the near-infrared range can also be used in this case. With sufficiently high gigawatt or preferably terawatt intensities per square centimetre, direct photoablation processes take place in which the molecular bonds are broken and no significant heat input into the surroundings occurs. This applies, in particular, to ultrashort pulses in the pico- and femtosecond range. It is known from K. König et al. (Opt. Lett. 26, 819 (2000)) that a short-pulse laser in the near-infrared range can be used to cut chromosomes in the cell nucleus, without damaging the surrounding cell. The minimum cut width is in this case 100 nanometres.

It is an object of the present invention to provide a method for carrying out electrical measurements on biological membrane bodies. This method is advantageously suitable for studying a large number of biological membrane bodies, and it is automatable and parallelizable. The conductive access to the biological membrane body, which is needed for the electrical measurement, is in this case intended to extend through the substrate and the biological membrane body lying on it, and to be made in situ, that is to say without previously detaching the biological membrane body from the substrate, by means of a laser ablation process.

A prerequisite for the invention is that the biological membrane body to be studied is located on a substrate. Suitable substrates are all thin layers of a material to which the biological membrane body can adhere. Suitable substrates are, for example, films of plastic, for example made of polyimide, silicone, polydimethyl siloxane, polycarbonate, polymethyl methacrylate (PMMA), polytetrafluoroethylene (PTFE), ABS (acrylonitrile/butadiene/styrene), polyamide (PA), polyethylene (PE), polyethylene terephthalate (PET), polybutylene terephthalate (PBT), polypropylene, polystyrene, or substrates which contain inorganic materials, for example borosilicate thin glass, quartz glass, silicon, silicon oxide, silicon nitrite, aluminium oxide, CVD diamond or ceramic. It is preferable to use non-conductive materials which have a dielectric constant of less than ∈=20, preferably less than ∈=10. The substrates have a thickness of between 10 μm and 500 μm, preferably between 30 μm and 200 μm. Optionally, the surface of the substrate is to be provided, for adhesion of cells or other biological membrane bodies, with a suitable coating which promotes attachment of the cells or other biological membrane bodies to be studied. To that end, a wide range of coatings have been described in the literature. Examples include proteins such as collagen, laminin or cadherin, antibodies, synthetic polylysine, lectins such as concanavalin A, or polysaccharides such as chitosan. Alternatively, the surface of the substrate is for this purpose to be hydrophilized, for example by a plasma treatment, for example in an argon plasma, so that it is attractive to biological membranes.

Biological membrane bodies in the context of the invention are, for example, living cells. This includes cells which have been isolated from living tissue by dissociation (primary cultures). It also includes cells which are kept in culture as established cell lines, for example CHO cells, HEK cells, NIH3T3 cells, HeLa cells but also transiently transfixed cells or primary cells. Biological membrane bodies in the context of the invention are furthermore artificially produced membrane bodies in which, for example, a lipid double layer encloses a limited volume of an aqueous medium (vesicle). These membrane bodies then advantageously contain at least one biological component, for example a polypeptide incorporated in the lipid double layer, a membrane-integrated enzyme, an ion channel or a G-protein-coupled receptor. Biological membrane bodies in the context of the invention may also be bacterial cells, fungal cells or cells of other single-celled or multi-celled organisms. Biological membrane bodies in the context of the invention are, for example, also protoplasts of fungal cells and plant cells as well as biological membrane bodies which—for example synaptosomes—have been produced by cleavage or purification from the membranes of living organisms, or which have been obtained by purifying such preparations with synthetic lipid vesicles.

A region of the substrate with at least one biological membrane body located on it is positioned in an optical instrument, for example an inverse or upright microscope, a confocal scanning laser microscope or another optical instrument in which the biological membrane bodies can be individually observed or identified, in such a way that a desired biological membrane body is sited centrally with respect to the optical axis of the illumination beam path. By using suitable optics, for example fibre coupling, the optical instrument is supplied with concentrated electromagnetic radiation, for example a laser beam, in such a way that the focus of the radiation is located on the lower side of the substrate, that is to say on the side facing away from the biological membrane body.

In one case of the present invention, the laser beam is in this case focused from below onto the lower side of the substrate by using suitable optics, for example a microscope objective with a high numerical aperture. This laser is a high-power laser, preferably a pulse laser, particularly preferably a UV pulse laser or a short-pulse laser, particularly preferably an ultrashort-pulse laser in the near infrared range. By focusing this laser beam, an intensity of more than one gigawatt per square centimetre, preferably terawatt per square centimetre, is achieved at the focus on the lower side of the substrate.

In another case of the present invention, the laser beam is focused from above onto the lower side of the substrate, through the biological membrane body and the substrate. This laser is a high-power laser, preferably a pulse laser or a short-pulse laser in the near infrared range, so that an interaction of the laser beam with the substrate and the biological membrane body takes place only with the very high intensities at the focus of the laser beam. This interaction advantageously involves multiphoton processes, that is to say processes which occur only when two or more photons interact simultaneously with the biological membrane body and the substrate. From R. Williams et al. (Curr. Opin. Chem. Biol.5, 603 (2001)), it is known that the intensity of the laser beam is so low outside the focus that no multiphoton processes occur, and little or no interaction takes place between the laser beam and the material.

In both described cases of the present invention, the substrate is then structured by the laser beam so as to form an access to the interior of the biological membrane body which is suitable for electrical measurements. In this case, the substrate is eroded by the laser in a narrowly bounded volume region starting from the lower side, that is to say the side facing away from the biological membrane. The erosion involves a laser ablation process, in which a channel is made in the substrate, preferably with a trapezoidal or quasi-trapezoidal cross section, the channel beginning relatively wide on the lower side of the substrate and then tapering inwards, so that the said channel has a diameter of advantageously at least 200 nm and at most 10 μm, but preferably a diameter of at least 500 nm and at most 5 μm, although more particularly preferably a diameter of at least 1 μm and at most 5 μm on the substrate side facing towards the said biological membrane body.

On the lower side of the substrate, the channel has a diameter of from 10 micrometres to one millimetre, preferably from 20 micrometres to 200 micrometres. The channel then terminates from 10 to 500 nanometres, preferably from 100 to 200 nanometres below the cell surface. In order to achieve corresponding widening of the channel on the lower side of the substrate, the substrate as well as the biological membrane body being observed may need to be moved by suitable micromanipulators. Alternatively, the laser beam may also be moved by means of deviating mirrors or other suitable deviating units. Confocal laser scanning microscopes are particularly suitable for this, since in this case the corresponding deviating units are already integrated in the microscope.

In order to accelerate the ablation process, or to increase its precision, the substrate may be doped with suitable absorbers, the absorbers needing to be matched to the wavelength of the laser beam which is used. In order to control the structuring speed of the ablation process, the absorber concentration may also be varied in the form of a gradual transition, with a high absorber concentration on the lower side of the substrate and a low absorber concentration on the upper side of the substrate. The structuring process may furthermore be accelerated by using pulse lasers with a high repetition rate, preferably with a repetition rate in the kilohertz range and, particularly preferably, with a repetition rate in the megahertz range.

When selecting the laser and the laser parameters, care was taken to ensure that only very minor heat input into the surrounding material occurs. This is preferably achieved by using short-pulse lasers with very short pulses, preferably with pulses shorter than one nanosecond and, particularly preferably, with pulses shorter than one picosecond.

In one case of the present invention, the channel in the substrate is distinguished by the fact that the inner walls are smooth.

After the channel which is open on one side has been formed, the substrate is placed in contact with an electrode system on the lower side, the side facing away from the biological membrane body. In particular, the channel which is open on one side is filled with a suitable electrolyte solution, which is compatible with the composition of the interior of the biological membrane body and with the intended purposes of the study (intracellular solution).

After contact has been made, the container which encloses the electrode system ends flush with the lower side of the substrate in an electrically leaktight fashion, in which case the lower side of the substrate may sometimes to be coated suitably, for example with silicone, petroleum jelly or another material.

In another case of the present invention, the side facing away from the biological membrane body is firstly brought into contact with the electrode system and the described ablation process is subsequently carried out.

For carrying out electrical measurements in accordance with this invention, it is necessary to produce a closed circuit, as is also employed for conventional patch-clamp measurements. This means that the electrolyte solution which communicates with the interior of the channel on the lower side of the substrate, as well as the electrolyte solution which is located on the upper side of the substrate and which encloses the biological membrane body, are respectfully connected by a suitable electrical contact to the input pole of a suitable electrical measurement amplifier, whereas no other electrical connection exists between these two electrolyte solutions other than via the biological membrane body to be measured and the channel leading to it. Silver/silver chloride electrodes, as already described for a long time in the literature, are advantageously used for the electrical contact with the electrolyte solution. The experimental arrangement comprises a container with an electrolyte solution and an electrode, as well as methods for pressure regulation and the necessary electronics for read-out of patch-clamp signals.

One form of the measuring arrangement according to the invention is advantageously configured in such a way that the container which encloses the electrode system is sufficiently transparent for the laser radiation which is used, so that it is possible to focus the laser beam through the electrode system onto the upper side of the substrate. By means of one or more laser pulses, it is then possible to erode the remaining substrate layer, for example. The seal resistance, that is to say the series resistance between the two electrolyte solutions on the substrate and in the interior of the channel, can subsequently be measured. Optionally, the necessary seal resistance in the megaohm or preferably gigaohm range can be made by applying a reinforcing negative pressure. An alternative would be to exert a pressure on the biological membrane body by using the laser beam as optical tweezers. The resistance is generally registered electrically, as is also done for patch-clamp measurements. Subsequently, the cell membrane which lies directly above the channel opening in the substrate can then be opened by means of one or more laser pulses.

In another form of the measuring arrangement according to the invention, the laser beam is focused from above through the biological membrane body, onto the remaining substrate layer directly below the biological membrane body, and this is eroded by means of one or more laser beams. The seal resistance between the biological membrane and the substrate is subsequently analysed. Optionally, the necessary seal resistance in the megaohm or preferably gigaohm range is produced by applying a reinforcing negative pressure. An alternative would be to exert a pressure on the biological membrane body by using the laser beam as optical tweezers. The resistance is generally registered electrically, as is also done for patch-clamp measurements. Subsequently, the cell membrane which lies directly above the channel opening in the substrate is then opened by means of one or more laser pulses.

In another form of the measuring arrangement according to the invention, the remaining substrate layer below the biological membrane body is eroded at the same time as the immediately adjacent membrane of the biological membrane body is opened.

For the subsequent electrical measurements, standard components of a patch-clamp measuring setup are used in the measuring arrangement according to the invention. All contemporary methods for electrophysiological studies can then be used. Examples of these include measuring the membrane potential of the biological membrane body, voltage-clamp measurement, current-clamp measurement, or measuring the membrane capacitance. Any desired substances relevant to the study may be added to the bath solution and hence delivered to the membrane of the biological membrane body, so that the effect on the electrical properties of the biological membrane body can be measured, as in conventional patch-clamp experiments. In particular, however, any desired substances may also be introduced into the interior of the biological membrane body through the channel, for example messenger substances, dyes, fluorescent dyes, chelating agents or other substances of biological or metrological interest. Optical measurements on the biological membrane body are hence also possible. Since, in conventional patch-clamp measurements, this channel corresponds to the interior of the patch pipette, for which it is only possible to change solutions with extra work, this constitutes an additional advantage of the measuring arrangement according to the invention.

If the access to the interior of the biological membrane body becomes obstructed by particles in the course of the measurement, then the access can be re-opened by means of new laser pulses. Owing to the setup which is used, simultaneous optical observation of the biological membrane bodies is also possible. The described method can furthermore be used for all adherent cells, and it is not restricted to culture cell lines.

The duration of the overall ablation process depends on the laser system which is used and on the substrate materials. When using a short-pulse laser in the near infrared range with a high repetition rate, the method according to the invention for making the electrically conductive access can be carried out in less than one second.

The method according to the invention is easy to automate. The biological membrane bodies can be selected by means of suitable image processing, and the substrate can be automatically positioned suitably by micromanipulators. Suitable cells can be identified by means of an optical method (in which, for example, experiments are carried out with an ion channel and green fluorescent protein (GFP) together). This corresponds to the integration of a fluorescence-activated cell sorter (FACS) into the described setup.

The ablation process can be automated by using computer-controllable lasers. The image processing may optionally control the ablation process. A control loop for controlling the ablation process, however, can also be produced by using the patch-clamp circuit.

The present invention can be parallelized, for example by correspondingly preparing all the chambers of a microtitration plate, for example with 96, 384 or 1536 openings, so that each individual one constitutes an above-described experimental setup, together with the miniaturized electrode system. The method according to the invention is then performed either sequentially or in parallel group-wise on the individual measurement chambers. To that end, corresponding beam-splitter optics and beam-guide components are employed. Inter alia, multi-channel amplifier systems are to be used for read-out of the signals, as are known from the MEA (multi-electrode array) technique or the detectors in high-energy physics. The invention is therefore well suited for use in industrial active-agent research with high or very high throughput.

One form of the measuring arrangement according to the invention makes it possible to re-close channels in the substrate, for example by using the laser beam which locally fuses the edge region of the channel. By means of this, it is possible to study a plurality of neighbouring membrane bodies on a substrate in succession.

The method according to the invention is distinguished over other methods for automating electrophysiological measurements by the fact that no positioning of the membrane bodies is needed, and that the channels which are needed for the method are made in situ in the substrate.

The invention relates to a method for measuring electrical signals from biological membrane bodies, characterized in that, below a biological membrane body located on a suitable substrate, a channel is made in the said substrate, the channel is filled with an electrolytic liquid, and an electrical access to the said membrane body is hence obtained.

The invention also relates to a method for measuring electrical signals from biological membrane bodies, characterized in that a respective channel is in each case made below at least two biological membrane bodies located on the same substrate, the channel is respectively filled with an electrolytic liquid, and an electrical access to the said membrane body is hence obtained.

The invention furthermore relates to a method for measuring electrical signals from biological membrane bodies, characterized in that, below a biological membrane body located on a suitable substrate, a channel is made in the said substrate, the channel is filled with an electrolytic liquid, and an electrical access to the said membrane body is hence obtained, the said substrate containing an electrically non-conductive material, whose dielectric constant is less than ∈=20.

The invention furthermore relates to a method for measuring electrical signals from biological membrane bodies, characterized in that, below a biological membrane body located on a suitable substrate, a channel is made in the said substrate, the channel is filled with an electrolytic liquid, and an electrical access to the said membrane body is hence obtained, an opening being made in the membrane body in the region covering the channel.

The invention furthermore relates to a method for measuring electrical signals from biological membrane bodies, characterized in that, below a biological membrane body located on a suitable substrate, a channel is made in the said substrate, the channel is filled with an electrolytic liquid, and an electrical access to the said membrane body is hence obtained, the said substrate containing thin glass, silicon, polyimide, silicone, polydimethyl siloxane, polycarbonate, polymethyl methacrylate (PMMA), polytetrafluoroethylene (PTFE), ABS (acrylonitrile/butadiene/styrene), polyamide (PA), polypropylene, polystyrene, polyethylene (PE), polyethylene terephthalate (PET), polybutylene terephthalate (PBT), silicon nitrite, aluminium oxide, silicon oxide, or CVD diamond.

The invention furthermore relates to a method for measuring electrical signals from biological membrane bodies, characterized in that, below a biological membrane body located on a suitable substrate, a channel is made in the said substrate, the channel is filled with an electrolytic liquid, and an electrical access to the said membrane body is hence obtained, the biological membrane body being an oocyte, a biological cell, a culture cell, a primary cell, a vesicle, or a fragment of a lipid double layer.

The invention furthermore relates to a method for measuring electrical signals from biological membrane bodies, characterized in that, below a biological membrane body located on a suitable substrate, a channel is made in the said substrate, the channel is filled with an electrolytic liquid, and an electrical access to the said membrane body is hence obtained, the said the channel having a diameter of at least 200 nanometres and at most 10 micrometres on the substrate side facing towards the said biological membrane body.

The invention furthermore relates to a method for measuring electrical signals from biological membrane bodies, characterized in that, below a biological membrane body located on a suitable substrate, a channel is made in the said substrate, the channel is filled with an electrolytic liquid, and an electrical access to the said membrane body is hence obtained, said channel having a conical cross section, the narrower cross section being located on the substrate side facing towards the biological membrane body.

The invention furthermore relates to a method for measuring electrical signals from biological membrane bodies, characterized in that, below a biological membrane body located on a suitable substrate, a channel is made in the said substrate, the channel is filled with an electrolytic liquid, and an electrical access to the said membrane body is hence obtained, the said channel being made by using a laser.

The invention furthermore relates to a method for measuring electrical signals from biological membrane bodies, characterized in that, below a biological membrane body located on a suitable substrate, a channel is made in the said substrate, the channel is filled with an electrolytic liquid, and an electrical access to the said membrane body is hence obtained, the said channel being subsequently re-closed by using a laser.

The invention furthermore relates to a method for measuring electrical signals from biological membrane bodies, characterized in that, below a biological membrane body located on a suitable substrate, a channel is made in the said substrate, the channel is filled with an electrolytic liquid, and an electrical access to the said membrane body is hence obtained, the laser beam striking the substrate on the side facing towards the biological membrane body.

The invention furthermore relates to a method for measuring electrical signals from biological membrane bodies, characterized in that, below a biological membrane body located on a suitable substrate, a channel is made in the said substrate, the channel is filled with an electrolytic liquid, and an electrical access to the said membrane body is hence obtained, the laser beam striking the substrate on the side facing away from the biological membrane body.

The invention furthermore relates to a device for measuring electrical signals from biological membrane bodies, containing a laser, a suitable mount for the substrate and an electrical measuring instrument, the said device being suitable for carrying out at least one of the above methods according to the invention. The invention furthermore relates to a method for determining the biological activity of membrane-integrated polypeptides, characterized in that, below a biological membrane body located on a suitable substrate, a channel is made in the said substrate, the channel is filled with an electrolytic liquid, and an electrical access to the said membrane body is hence obtained, and electrical measurements are subsequently performed on the said membrane body through the channel, the result of the electrical measurements being dependent on the activity of the membrane-integrated polypeptide.

The invention furthermore relates to the above method, the said polypeptide being an ion channel or a G-protein-coupled receptor.

The invention furthermore relates to a method for identifying active agents, characterized in that, below a biological membrane body located on a suitable substrate, a channel is made in the said substrate, the channel is filled with an electrolytic liquid, and an electrical access to the said membrane body is hence obtained, and electrical measurements are subsequently performed on the said membrane body, in each case in the presence of a respective test substance, the test substances constituting active agents being those for which the measured electrical signals differ significantly at different concentrations of the test substance.

The invention furthermore relates to active agents found by the above method, as well as to their use for producing medicaments.

“Active agents” in the context of the invention are substances which can influence one or more biological activities or properties of an organism, a cell or a polypeptide. By way of example, pharmaceutical active agents will be mentioned here.

“Test substances” in the context of the invention are all substances which are used, have been used and/or can be used in one of the methods above or below.

“Significant” in the context of the invention means: the measurement values differ by amounts which are equal to or greater than the statistical measurement uncertainty. The statistical measurement uncertainty can be determined by repeated measurements under constant conditions. It is then equal to the standard deviation in this series of measurements.

The invention furthermore relates to a method for screening substance libraries with respect to their being inhibitors or activators of a membrane-integrated polypeptide, characterized in that a method is carried out on a biological membrane body, containing the said membrane-integrated polypeptide, in the presence of a respective single substance from the substance library, the test substances constituting potential inhibitors or activators of the membrane-integrated polypeptide being those whose presence and/or concentration has a significant effect on the activity of the membrane-integrated polypeptide.

The invention furthermore relates to the activators or inhibitors found by the above method, as well as to their use for producing medicaments.

### DESCRIPTION OF THE DRAWINGS

FIG. 1 is a schematic representation, not true to scale and not accurate in detail, of the setup for the described method, consisting of the laser beam 1, a substrate 2 and the biological membrane body 3. In one case, the laser beam 1 comes from below (left-hand image), and in the other case it comes from above (right-hand image).

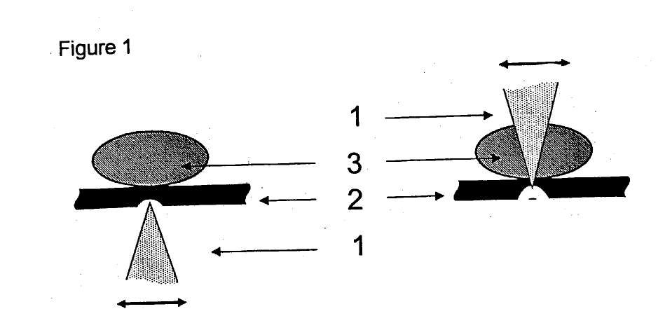

FIG. 1 is a schematic representation, not true to scale and not accurate in detail, of the setup for an electrical measurement, consisting of a substrate 2, the biological membrane body 3 and the electronic circuit for the electrical measurement 4.

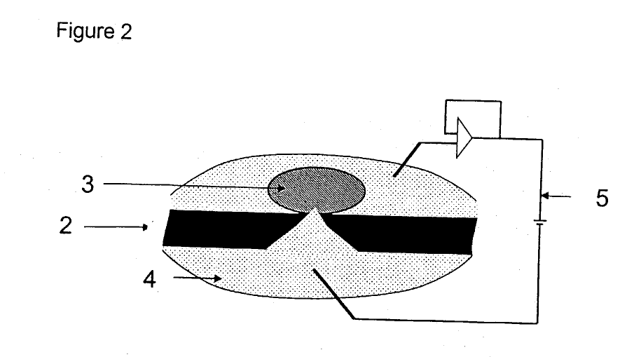

### EXAMPLES

The method is especially suitable for use in active-agent research, as a replacement for conventional and automated patch-clamp techniques and as a portable biosensor, for example in environmental analysis. Examples of measuring arrangements and fields of application thereof will be described below.

The measuring arrangement according to the invention has, for example, at least one substrate, and suitable biological membrane bodies for electrical measurements are located on this substrate.

On the lower side of the substrate, there is, for example, a device which contains at least one electrode and has a compartment suitable for holding liquid. The measuring arrangement furthermore has the corresponding electronics and software for read-out, representation and evaluation of the measurement signals.

Optionally, the measuring arrangement has a device with which a laser beam can be focused onto the substrate below the biological membrane body and onto the membrane immediately above the substrate.

Advantageously, the measuring arrangement has means on one side, or preferably on both sides, which permit liquid delivery, liquid storage or replacement of liquids.

### Example 1

CHO cells are cultivated on a substrate made of borosilicate thin glass (Schott display glass D 263 T, thickness 50 micrometres), so that the density of the cells is such that the individual cells lying in isolation can be identified without difficulty. The substrate is bounded by an elevation, so that the substrate surface and the elevation form a flat vessel, which is filled with a suitable nutrient or electrolyte solution for the cells.

The substrate with adherent cells is positioned on the stage of an upright laser scanning a microscope (Zeiss LSM 510). A suitably appearing cell is selected by means of conventional direct-light microscopy and is positioned centrally with respect to the optical axis by means of the microscope stage. An electrode is also introduced into the solution, so that it makes an electrical contact with the solution but does not interfere with the beam path.

The stage of the microscope is modified in such a way that an electrode system can be brought into contact with the substrate even from below in the region of the optical axis of the system. This involves a small vessel, which is filled with the electrolyte solution and in which an electrode is located. The side of the vessel facing upwards is open, and the other side is closed or joined to a suitable system for adjusting the pressure and for replacing solutions or for delivering substances. The electrode is connected to the amplifier and the measurement electronics. The side the vessel which points upwards is open and the edge may, for example, be coated with silicone in order to achieve a mechanical seal against the lower side of the substrate.

A high-power short-pulse laser in the near infrared range (Spectra Physics Mai Lai) is applied to the laser scanning microscope and is focused through the cell onto the lower side of the substrate by means of an objective with a large working distance. Within the focal plane, a power in excess of 300 milliwatts is obtained for this laser. The substrate is then eroded by the laser beam, beginning on the lower side, so that a channel is formed which has a diameter of 100 micrometres on the lower side and a diameter of one micrometre immediately below the cell. Before the channel emerges on the upper surface of the substrate, the electrode system is brought into contact with the lower side of the substrate, so that an electrically leaktight connection is formed at the contact between the cylinder and the lower side of the glass substrate. Using one or more laser pulses, the remaining substrate material below the cell is then eroded, so that the cell membrane comes into direct contact with the electrode system.

For intracellular measurements, the cell membrane is then opened in the region of the channel opening by using one or more laser pulses, and this is detected by means of corresponding electronic signals. All contemporary methods for electrophysiological studies can be used for the subsequent electrical measurements. Besides the electrical measurements, simultaneous optical observation of the cells is also possible.

If the cell access becomes obstructed by particles in the course of the measurement, then the access is re-opened by means of one or more laser pulses.

### Example 2

CHO cells are cultivated on a substrate made of borosilicate thin glass (Schott display glass D 263 T, thickness 50 micrometres), so that the density of the cells is such that the individual cells lying in isolation can be identified without difficulty. The substrate is bounded by an elevation, so that the substrate surface and the elevation form a flat vessel, which is filled with a suitable nutrient or electrolyte solution for the cells.

The substrate with adherent cells is positioned on the stage of an upright laser scanning a microscope (Zeiss LSM 510). A suitable cell is then selected by means of conventional direct-light microscopy and is positioned centrally with respect to the optical axis by means of the microscope stage. A bath electrode is also introduced into the solution, so that it lies as close as possible to the selected cell.

The stage of the microscope is modified in such a way that an electrode system can be brought into contact with the substrate from below in the region of the optical axis of the system. This involves a small vessel, which is filled with the electrolyte solution and in which an electrode is located. The lower side of the vessel is closed by a glass plate of optical quality, which is transparent for the laser radiation being used, so that the laser beam can be focussed through the electrode system onto the substrate material. The lateral opening of the vessel may be connected to a suitable system for adjusting the pressure and for replacing solutions or for delivering substances, or it is closed. The electrode is connected to the amplifier and the measurement electronics. The upper side the vessel is open and the edge may, for example, be coated with silicone in order to achieve a mechanical seal against the lower side of the substrate.

A high-power short-pulse laser in the near infrared range (Spectra Physics Mai Lai) is applied to the laser scanning microscope and is focused through the cell onto the lower side of the substrate by means of a microscope objective. Within the focal plane, a power in excess of 300 milliwatts is obtained for this laser. The substrate is then eroded by the laser beam, beginning on the lower side, so that a channel is formed which has a diameter of 100 micrometres on the lower side and a diameter of one micrometre immediately below the cell. Before the channel emerges on the upper surface of the substrate, the electrode system is brought into contact with the lower side of the substrate, so that an electrically leaktight connection is formed at the contact between the cylinder and the lower side of the glass substrate. The objective is replaced by an objective with a longer working distance, and the laser beam is focused by it through the electrode system onto the upper side of the substrate. Using one or more laser pulses, the remaining substrate material below the cell is then eroded, so that the cell membrane comes into direct contact with the electrode system.

For intracellular measurements, the cell membrane is then opened in the region of the channel opening by using one or more laser pulses, and this is detected by means of corresponding electronic signals. All contemporary methods for electrophysiological studies can be used for the subsequent electrical measurements. Besides the electrical measurements, simultaneous optical observation of the cells is also possible.

If the cell access becomes obstructed by particles in the course of the measurement, then the access is re-opened by means of one or more laser pulses.

### Example 3

As Example 2, a high-power UV short-pulse laser being applied to the laser scanning microscope and focused onto the lower side of the substrate by means of a microscope objective.

### Example 4

Corresponding to Examples 1 to 3, Schott display glass AF 45, 50 micrometres thick, being used.

### Example 5

Corresponding to Examples 1 to 4, the electrode system being brought into contact with the substrate before the beginning of the ablation process.

### Example 6

Corresponding to Examples 1 to 4, the cell membrane not being opened in the region of the channel opening.

1. Method for measuring electrical signals from biological membrane bodies, comprising, below a biological membrane body located on a suitable substrate, making a channel in said substrate, filling the channel with an electrolytic liquid, and thereby obtaining an electrical access to said membrane body.

2. Method according to claim 1, in which a respective channel is in each case made below at least two biological membrane bodies located on the same substrate.

3. Method according to claim 1, in which the said substrate contains an electrically non-conductive material, whose dielectric constant is less than ∈=20.

4. Method according to claim 1, in which an opening is made in the membrane body in a region covering the channel.

5. Method according to claim 1, in which the said substrate contains thin glass, silicon, polyimide, silicone, polydimethyl siloxane, poly carbonate, polymethyl methacrylate (PMMA), polytetrafluoroethylene (PTFE), ABS (acrylonitrile/butadiene/styrene), polyamide (PA), polypropylene, polystyrene, polyethylene (PE), polyethylene terephthalate (PET), polybutylene terephthaiate (PBT), silicon nitrite, aluminium oxide, silicon oxide, or CVD diamond.

6. Method according to claim 1, in which the biological membrane body is
i) an oocyte,
ii) a biological cell,
iii) a culture cell,
iv) a primary cell,
v) a vesicle, or
vi) a fragment of a lipid double layer.

7. Method according to claim 1, in which said channel has a diameter of at least 200 nm and at most 10 μm on a substrate side facing towards said biological membrane body.

8. Method according to claim 1, in which said channel has a conical cross section, a narrower cross section being located on a side of the substrate facing towards the biological membrane body.

9. Method according to claim 1, in which said channel is made by using a laser.

10. Method for measuring electrical signals from biological membrane bodies, in which the method according to claim 9 is carried out first, and then the channel is subsequently re-closed using a laser.

11. Method according to claim 9, in which the laser beam strikes the substrate on a side facing towards the biological membrane body.

12. Method according to claim 9, in which the laser beam strikes the substrate on a side facing away from the biological membrane body.

13. Device for measuring electrical signals from biological membrane bodies, comprising
i) a laser,
ii) a suitable mount for a substrate,
iii) and an electrical measuring instrument.

14. Method for determining the biological activity of membrane-integrated polypeptides, comprising, below a biological membrane body located on a suitable substrate, making a channel in said substrate, filling the channel with an electrolytic liquid, and thereby obtaining an electrical access to said membrane body, and subsequently performing electrical measurements on said membrane body through the channel, a result of the electrical measurements being dependent on the activity of the membrane-integrated polypeptide.

15. Method according to claim 14, in which said polypeptide is an ion channel or a G-protein coupled receptor.

16. Method for identifying active agents, comprising, below a biological membrane body located on a suitable substrate, making a channel in the said substrate, filling the channel with an electrolytic liquid, and thereby obtaining an electrical access to the said membrane body, and subsequently performing electrical measurements on said membrane body, in each case in the presence of a respective test substance, the test being one which yields significantly different measured electrical signals at different concentrations of the test substance.

17. Method for screening substance libraries with respect to their being inhibitors or activators of a membrane-integrated polypeptide, comprising carrying out a method according to claim 14 on a biological membrane body containing said membrane-integrated polypeptide, in the presence of a respective single substance from the substance library, the test substance constituting potential inhibitors or activators of the membrane-integrated polypeptide being those whose presence and/or concentration has a significant effect on the activity of the membrane-integrated polypeptide.
